(19) United States
(12) Patent Application Publication
(10) Pub. No.: US 2021/0113637 A1
(43) Pub. Date: Apr. 22, 2021

(54) IMMUNOMODULATORY COMPOSITIONS AND USES THEREOF

(71) Applicant: INTEGRIA HEALTHCARE (AUSTRALIA) PTY LIMITED, New South Wales (AU)

(72) Inventors: Joe TIRALONGO, Queensland (AU); Brody MALLARD, Queensland (AU); David LEACH, Queensland (AU); Hans WOHLMUTH, Queensland (AU)

(21) Appl. No.: 17/256,383

(22) PCT Filed: Apr. 4, 2019
(86) PCT No.: PCT/AU2019/050295

§ 371 Date: Dec. 28, 2020

(30) Foreign Application Priority Data
Jun. 29, 2018 (AU) 2018902345

(51) Int. Cl. A61K 36/074; A61K 36/07; A61K 31/716; A61P 37/04

(52) U.S. Cl. CPC A61K 36/074; A61K 36/07; A61K 2236/331; A61P 37/04; A61K 2236/333; A61K 31/716

(57) ABSTRACT

The invention relates to immunomodulatory compositions comprising one or more extracts from medicinal fungi, and the use of such compositions in the treatment or prevention of conditions or diseases associated with immunological dysfunction, and/or to provide one or more health benefits to an animal subject. In certain embodiments, the invention relates to compositions comprising multiple extracts from a number of medicinal mushrooms, and the use of such compositions to provide one or more health benefits to a subject in need thereof.

### FIELD OF INVENTION

The invention relates to immunomodulatory compositions comprising one or more extracts from medicinal fungi, and the use of such compositions in the treatment or prevention of conditions or diseases associated with immunological dysfunction, and/or to provide one or more health benefits to an animal subject. In certain embodiments, the invention relates to compositions comprising multiple extracts from a number of medicinal mushrooms, and the use of such compositions to provide one or more health benefits to a subject in need thereof.

### BACKGROUND OF THE INVENTION

The treatment or prevention of diseases and conditions associated with the immune system and/or aberrant immune function, together with the amelioration of symptoms associated with immune dysfunction (such as immunodeficiencies or autoimmunity) remain significant medical and economic problems.

Medicinal mushrooms are comprised of a variety of bioactive compounds with immunomodulatory activities, such as polysaccharides (in particular β-D-glucans), polysaccharopeptides (PSP), phenolic compounds, proteins, lipid components, and terpenoids. The immunomodulatory effects of these bioactive compounds have been reported to be derived from the immune system's ability to recognise these compounds with a resultant activation of immune cells; macrophages, lymphocytes, dendritic cells (DCs) and natural killer (NK) cells.

These immunomodulatory effects and subsequent potential for therapeutic benefits are generating a renewed interest in the scientific investigation of medicinal mushrooms. Despite this interest and a significant body of research, there remains a need for effective immunomodulatory compositions derived from medicinal mushrooms or extracts therefrom, particularly those which are capable of eliciting a pro-inflammatory or anti-inflammatory cytokine response.

It is an object of the invention to provide one or more immunomodulatory compositions, such as one or more compositions capable of eliciting an effect on the expression of pro-inflammatory and/or anti-inflammatory cytokines, and/or to at least provide the public with a useful choice.

### SUMMARY OF THE INVENTION

In a first aspect the invention relates to an immunomodulatory composition, the immunomodulatory composition comprising at least one extract from each of two or more medicinal fungi, wherein the composition elicits a synergistic immunomodulatory effect.

In a second aspect the invention relates to an immunomodulatory composition, the immunomodulatory composition comprising at least one extract from each of two or more medicinal fungi, wherein each of the fungal extracts comprises β-glucan and has a f3-glucan:total glucan ratio of at least about 0.6.

In a further aspect, the invention relates to a method of modulating the immune system in a subject in need thereof, the method comprising administering to the subject an effective amount of an immunomodulatory composition as described herein.

In a further aspect, the invention relates to a method of eliciting an immune response in a subject, the method comprising administering to the subject an effective amount of an immunomodulatory composition as described herein.

In another aspect, the invention relates to a method of treating a disease or condition associated with the immune system or of treating a disease or condition associated with immune dysfunction in a subject in need thereof, the method comprising administering to the subject an effective amount of an immunomodulatory composition as described herein.

In another aspect, the invention relates to a method of conferring a health benefit to a subject in need thereof, the method comprising administering to the subject an effective amount of an immunomodulatory composition as described herein.

In still a further aspect the invention relates to a method of reducing susceptibility to one or more infectious diseases in a subject in need thereof, the method comprising administering to the subject an effective amount of an immunomodulatory composition as described herein.

In another aspect the invention relates to the use of at least one extract from each of two or more medicinal fungi in the preparation of a medicament, wherein each of the fungal extracts comprises f3-glucan and has a f3-glucan:total glucan ratio of at least about 0.6.

In various embodiments the medicament is for use in modulating the immune system of a subject in need thereof, for use in eliciting an immune response in a subject in need thereof, for use in eliciting an immunostimulatory response in a subject in need thereof, or for conferring a health benefit to a subject in need thereof.

In various embodiments, the medicament is for eliciting an immune response in a subject, for treating a disease or condition associated with the immune system, for treating a disease or condition associated with immune dysfunction in a subject in need thereof, for conferring a health benefit to a subject in need thereof, or for reducing susceptibility to one or more infectious diseases in a subject in need thereof.

The invention further relates to a composition as described herein for use in modulating the immune system of a subject in need thereof, for use in eliciting an immune response in a subject in need thereof, for use in eliciting an immunostimulatory response in a subject in need thereof, or for conferring a health benefit to a subject in need thereof.

Any of the embodiments described herein may relate to any aspect of the invention.

In one embodiment, one or more of the medicinal fungi is a medicinal mushroom. In one 25 embodiment, each of the medicinal fungi is a medicinal mushroom.

In one embodiment, the medicinal mushroom is selected from the group comprising Reishi Ganoderma lucidum, Cordyceps sinensis, Maitake Grifola frondosa, Shiitake Lentinula edodes, Poria cocos, Lion's Mane Hericium erinaceus, Mesima Phellinus linteus, Turkey 30 Tail (Coriolus) Trametes versicolor, and Chaga Inonotus obliquus.

In one embodiment, the medicinal mushroom is selected from the group comprising Reishi, Shiitake, and Maitake.

In one embodiment the Reishi is Ganoderma lucidum. In one embodiment, the Shiitake is Lentinula edodes. In one embodiment the Maitake is Grifola frondosa.

In one embodiment the composition comprises at least one extract from each of Reishi, Shiitake, and Maitake.

In one embodiment, at least one of the extracts is an extract from fungal mycelium.

In one embodiment, at least one of the extracts is an extract of fungal fruiting bodies. For example, each of the extracts present in the composition is an extract of fungal fruiting bodies.

In one embodiment, at least one of the extracts is an extract of fungal fruiting bodies substantially free of mycelia or mycelial extracts. In one specifically contemplated embodiment, each of the extracts present in the composition is an extract of fungal fruiting bodies substantially free of mycelia or mycelial extracts.

In one embodiment, the composition is a pharmaceutical composition and comprises one or more pharmaceutically acceptable carriers.

In one embodiment, the composition comprises one or more extracts from fungal mycelium and fungal fruiting bodies.

In one embodiment, the composition comprises at least one extract from the fruiting 25 bodies of each of two or more medicinal fungi. For example, the composition comprises at least one extract from the fruiting body of each of Ganoderma lucidum, Lentinula edodes, and Grifola frondosa fungi.

In various embodiments, each of the fungal extracts comprises β-glucan, wherein the β-30 glucan comprises at least about 10% w/w of the total fungal polysaccharides present.

In various embodiments, one or more of the fungal extracts comprises less than about 5% w/w α-glucan. For example, one or more of the fungal extracts comprises less than about 5% w/w α-glucan with reference to the total fungal polysaccharides present in the extract.

In one embodiment, each of the fungal extracts comprises less than about 5% w/w α-glucan with reference to the total fungal polysaccharides present in each extract. In one embodiment, one or more of the fungal extracts is substantially free of α-glucan.

In one embodiment, the weight ratio of β-glucan: α-glucan in one or more of the fungal extracts, for example one or more of the fungal fruiting body extracts, is at least about 2:1, at least about 3:1, at least about 4:1, or is at least about 5:1. In another example, the weight ratio of β-glucan: α-glucan in one or more of the fungal extracts, for example one or more of the fungal fruiting body extracts, is at least about 6:1, at least about 7:1, at least about 8:1, at least about 9:1, at least about 10:1, at least about 15:1, at least about 20:1, or greater than about 20:1.

In one embodiment, the weight ratio of β-glucan: α-glucan in each of the fungal extracts, for example each of the fungal fruiting body extracts, is at least about 2:1, at least about 3:1, at least about 4:1, or is at least about 5:1. In another example, the weight ratio of β-glucan: α-glucan in each of the fungal extracts, for example each of the fungal fruiting body extracts, is at least about 6:1, at least about 7:1, at least about 8:1, at least about 9:1, at least about 10:1, at least about 15:1, at least about 20:1, or greater than about 20:1.

In one embodiment, the concentration of fungal β-glucan present in the composition is at least about five fold higher than the concentration of fungal α-glucan.

For example, the weight ratio of β-glucan: α-glucan in the composition is at least about 2:1, at least about 3:1, at least about 4:1, or is at least about 5:1. In another example, the weight ratio of β-glucan: α-glucan in the composition is at least about 6:1, at least about 7:1, at least about 8:1, at least about 9:1, at least about 10:1, at least about 15:1, at least about 20:1, or greater than about 20:1.

It will be appreciated that, unless the context clearly indicates otherwise, the term “weight ratio” refers to w/w, such that a weight ratio of 2:1 could equivalently be expressed as 2:1 (w/w).

In one embodiment, the combined fungal extracts present in the composition comprise less than about 5% w/w α-glucan.

In one embodiment, the composition comprises extracts, for example fruiting body extracts, from each of Maitake, Reishi, and Shiitake fungi.

In one embodiment, the composition consists of fruiting body extracts from each of each of Maitake, Reishi, and Shiitake fungi, optionally together with one or more pharmaceutically acceptable carriers. For example, the composition consists of fruiting body extracts from each of each of Maitake, Reishi, and Shiitake fungi, optionally together with one or more pharmaceutically acceptable carriers, wherein each of the fruiting body extracts is substantially free of mycelia or mycelial extracts.

In various embodiments, on a dry weight equivalent basis the Maitake:Reishi ratio is from about 1:4 to about 4:1, for example the Maitake:Reishi ratio is from about 1:3 to about 3:1, from about 1:2 to about 2:1, from about 1:1.5 to about 1.5:1, or about 1:1.

In one specifically contemplated embodiment on dry weight basis the Maitake:Reishi ratio is about 1:1.2.

In various embodiments, on a dry weight equivalent basis the Maitake:Shiitake ratio is from about 1:4 to about 4:1, for example the Maitake:Shiitake ratio is from about 1:3 to about 3:1, from about 1:2 to about 2:1, from about 1:1.5 to about 1.5:1, or about 1:1.

In one specifically contemplated embodiment on dry weight basis the Maitake:Shiitake 25 ratio is about 1:1.2.

In various embodiments, on a dry weight equivalent basis the Reishi:Shiitake ratio is from about 1:4 to about 4:1, for example the Reishi:Shiitake ratio is from about 1:3 to about 3:1, from about 1:2 to about 2:1, from about 1:1.5 to about 1.5:1, or about 1:1.

In one specifically contemplated embodiment on dry weight basis the Reishi:Shiitake ratio is 1:1.

In a further specifically contemplated embodiment, on a dry weight equivalent basis the Maitake:Reishi:Shiitake ratio is 1:1.2:1.2. For example, the composition comprises extracts of fruiting bodies from each of Maitake, Reishi, and Shiitake in an amount on a dry weight equivalent basis of fruiting bodies in the ratio of 1:1.2:1.2.

In one embodiment, one or more of the extracts are selected from the extracts presented in Table 1 herein.

In one embodiment, the fungal extracts comprise at least 50% w/w of the composition. For example, the fungal extracts comprise at least about 60% w/w, at least about 70% w/w, at least about 75% w/w, at least about 80% w/w, at least about 85% w/w, at least about 90% w/w, or more than about 90% w/w of the composition.

In one embodiment the synergistic immunomodulatory effect is a synergistic immunostimulatory effect. It will be appreciated that in the context of the present invention a synergistic effect, such as a synergistic effect mediated by a composition described herein, is an effect that is of greater magnitude than the sum of the effects elicited by the individual active constituents alone. It will further be appreciated that a synergistic immunomodulatory effect may be a synergistic pro-inflammatory effect or a synergistic anti-inflammatory effect.

Similarly, it will be appreciated on reading this specification that synergism, such as that embodied in a synergistic response, can be assessed and determined in a number of different ways, including by methods described and exemplified herein, such as the response additivity graph analyses and the curve-shift analyses well known in the art and described and exemplified herein.

In one embodiment the immunomodulatory effect is an increase in the expression of one or more pro-inflammatory cytokines. For example, the immunomodulatory effect is an increase in the expression of one or more pro-inflammatory cytokines by one or more immune cells, including one or more mammalian immune cells.

In one example, the immunomodulatory effect is an increase in the expression of one or more pro-inflammatory cytokines by one or more macrophages, one or more neutrophils, one or more dendritic cells, one or more CD4+ lymphocytes, one or more CD8+ lymphocytes, one or more NK cells, one or more mast cells, or one or more eosinophils.

In various embodiments, the one or more pro-inflammatory cytokines is selected from the group comprising IL-1, IL-1α, IL-6, and TNF-α.

Accordingly, in one embodiment the composition elicits a synergistic increase in the expression of one or more pro-inflammatory cytokines, for example by one or more macrophages. For example, the composition elicits a synergistic increase in the expression of one or more of IL-1α, IL-6, or TNF-α, such as a synergistic increase in the expression of IL-1α, IL-6, and TNF-α by one or more macrophages.

In one embodiment the immunomodulatory effect is a decrease in the expression of one or more anti-inflammatory cytokines. For example, the immunomodulatory effect is a decrease in the expression of one or more anti-inflammatory cytokines by one or more immune cells, including one or more mammalian immune cells.

In one example, the immunomodulatory effect is a decrease in the expression of one or more anti-inflammatory cytokines by one or more macrophages, one or more neutrophils, one or more dendritic cells, one or more CD4+ lymphocytes, one or more CD8+ lymphocytes, one or more NK cells, one or more mast cells, or one or more eosinophils.

In various embodiments, the one or more anti-inflammatory cytokines is selected from the group comprising IL-1Rα, IL-4, IL-10, IL-11, IL-13, and TNF-β. For example, the anti-inflammatory cytokine is IL-10.

Accordingly, in one embodiment the composition elicits a synergistic decrease in the expression of one or more anti-inflammatory cytokines, for example by one or more macrophages. For example, the composition elicits a synergistic decrease in the expression of IL-10, such as a synergistic decrease in expression of IL-10 by one or more macrophages.

In one embodiment, the composition elicits a synergistic increase in the expression of one or more pro-inflammatory cytokines, and a synergistic decrease in the expression of one or more anti-inflammatory cytokines.

In various embodiments, the one or more health benefits is selected from the group comprising: supporting healthy immune function, improving convalescence, reducing fatigue, improving respiratory function, reducing coughing, improving immune responses to infectious disease or decreasing susceptibility to infectious disease.

In various embodiments, the one or more health benefits is selected from the group comprising: an increase in one or more antioxidants, one or more antiatherosclerotic effects, one or more hepato-protective effects, one or more immunological benefits, one or more analgesic effects, one or more antimicrobial effects, one or more antiparasitic effects, one or more antifungal effects, one or more antiviral effects, one or more chemopreventive effects, one or more anticancer effects, one or more hypolipidemic effects, one or more antiatherogenic effects, one or more antiplatelet and/or antithrombotic effects, one or more anti-diabetic effects, one or more antihypertensive effects, one or more antinociceptive effects, one or more anti-asthma effects, one or more wound-healing effects, one or more food allergy effects, one or more antiaging effects, one or more hypoglycemic effects, one or more anti-arthritis effects, and one or more antiulcer effects, wherein one or more of the above potential therapeutic effects are derived from the immunomodulatory properties of the compositions described herein.

In certain embodiments, the subject is a mammal. The mammal may be of either sex and may be at any stage of development.

In certain embodiments, the subject is a human.

In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

As used herein the term “and/or” means “and” or “or”, or both.

As used herein “(s)” following a noun means the plural and/or singular forms of the noun.

The term “comprising” as used in this specification means “consisting at least in part of”. When interpreting statements in this specification which include that term, the features, prefaced by that term in each statement, all need to be present, but other features can also be present. Related terms such as “comprise” and “comprised” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7).

To those skilled in the art to which the invention relates, many changes in construction and widely differing embodiments and application of the invention will suggest themselves without departing from the scope of the invention as defined in the appended claims. The disclosures and the descriptions herein are purely illustrative and are not intended to be in any sense limiting.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

Embodiments of the invention will now be described, by way of example only, with reference to the accompanying drawings in which:

FIG. 1 is four graphs, each depicting triplicate repeats assessing the effect on cytokine expression of varying concentrations of Shiitake extract in ±LPS-stimulated human macrophages. A, TNF-α; B, IL-1α; C, IL-6; D, IL-10. Concentrations are represented by log value and range from 1 to 1,000 μg/ml. Control and Control+LPS dotted lines pass through the y-axis of the graph representing normal cytokine expression within the macrophages. Shiitake extract +LPS, solid line; Shiitake extract −LPS, dashed line; Control (+LPS only), horizontal dashed dotted line; Control (cells only), horizontal dotted line.

FIG. 2 is four graphs, each depicting triplicate repeats assessing the effect on cytokine expression of varying concentrations of Reishi extract in ±LPS-stimulated human macrophages. A, TNF-α; B, IL-1α; C, IL-6; D, IL-10. Concentrations are represented by log value and range from 1 to 1,000 μg/ml. Control and Control+LPS dotted lines pass through the y-axis of the graph representing normal cytokine expression within the macrophages. Reishi extract +LPS, solid line; Reishi extract −LPS, dashed line; Control (+LPS only), horizontal dashed dotted line; Control (cells only), horizontal dotted line.

FIG. 3 is four graphs, each depicting triplicate repeats assessing the effect on cytokine expression of varying concentrations of Maitake extract in ±LPS-stimulated human macrophages. A, TNF-α; B, IL-1α; C, IL-6; D, IL-10. Concentrations are represented by log value and range from 1 to 1,000 μg/ml. Control and Control+LPS dotted lines pass through the y-axis of the graph representing normal cytokine expression within the macrophages. Maitake extract +LPS, solid line; Maitake extract −LPS, dashed line; Control (+LPS only), horizontal dashed dotted line; Control (cells only), horizontal dotted line.

FIG. 4 is four graphs, each depicting triplicate repeats assessing the effect on cytokine expression of varying concentrations of multi-extract composition M18-13 in ±LPS-stimulated human macrophages. A, TNF-α; B, IL-1α; C, IL-6; D, IL-10. Concentrations are represented by log value and range from 1 to 1,000 μg/ml. Control and Control+LPS dotted lines pass through the y-axis of the graph representing normal cytokine expression within the macrophages. Multi-extract composition M18-13+LPS, solid line; multi-extract composition M18-13 −LPS, dashed line; Control (+LPS only), horizontal dashed dotted line; Control (cells only), horizontal dotted line.

FIG. 5 is four graphs, each depicting a nonlinear regression EC50 curve of the effect on cytokine expression of varying concentrations of Shiitake extract in ±LPS-stimulated human macrophages. A, TNF-α; B, IL-1α; C, IL-6; D, IL-10. Concentrations are represented by log value and range from 1 to 1,000 μg/ml. Shiitake extract +LPS, solid line; Shiitake extract −LPS, dashed line.

FIG. 6 is four graphs, each depicting a nonlinear regression EC50 curve of the effect on cytokine expression of varying concentrations of Reishi extract in ±LPS-stimulated human macrophages. A, TNF-α; B, IL-1α; C, IL-6; D, IL-10. Concentrations are represented by log value and range from 1 to 1,000 μg/ml. Reishi extract +LPS, solid line; Reishi extract −LPS, dashed line.

FIG. 7 is four graphs, each depicting a nonlinear regression EC50 curve of the effect on cytokine expression of varying concentrations of Maitake extract in ±LPS-stimulated human macrophages. A, TNF-α; B, IL-1α; C, IL-6; D, IL-10. Concentrations are represented by log value and range from 1 to 1,000 μg/ml. Maitake extract +LPS, solid line; Maitake extract −LPS, dashed line.

FIG. 8 is four graphs, each depicting a nonlinear regression EC50 curve of the effect on cytokine expression of varying concentrations of multi-extract composition M18-13 in ±LPS-stimulated human macrophages. A, TNF-α; B, IL-1α; C, IL-6; D, IL-10. Concentrations are represented by log value and range from 1 to 1,000 μg/ml. multi-extract composition M18-13+LPS, solid line; multi-extract composition M18-13 −LPS, dashed line.

FIG. 9 is eight graphs, each depicting the effect-based response additivity for cytokine expression induced by individual mushroom extracts and the multi-extract composition M18-13 in LPS stimulated and non-stimulated human macrophages. The dashed line represents the expected additive effect and represents the level of expression expected if the response derived from the multi-extract composition (M18-13) was simply the sum of the individual responses of its component extracts. A response above the additive line indicates a potential synergistic effect between the extracts and beneath the line a potential antagonistic effect. A TNF-α in LPS stimulated macrophages, C IL-1α in LPS stimulated macrophages, E IL-6 in LPS stimulated macrophages, and G IL10 in LPS stimulated macrophages; B TNF-α in non-LPS stimulated macrophages, D IL-1α in non-LPS stimulated macrophages, F IL-6 in non-LPS stimulated macrophages, and H IL-10 in non-LPS stimulated macrophages.

FIG. 10 is eight graphs, each depicting a curve shift analysis for cytokine expression induced by individual mushroom extracts and the multi-extract composition M18-13 in LPS stimulated and non-stimulated human macrophages. The dose-effect curve for multi-extract composition M18-13 (▾ solid black line) is compared to the expected additive effect of the individual extract responses (♦ dashed black line), and the individual mushroom extracts Shiitake (M18-2) (◯ grey line), Reishi (M18-1) (□ grey line) and Maitake (M18-3) (⋄ grey line). A TNF-α in LPS stimulated macrophages, C IL-1α in LPS stimulated macrophages, E IL-6 in LPS stimulated macrophages, and G IL10 in LPS stimulated macrophages; B TNF-α in non-LPS stimulated macrophages, D IL-1α in non-LPS stimulated macrophages, F IL-6 in non-LPS stimulated macrophages, and H IL-10 in non-LPS stimulated macrophages.

### DETAILED DESCRIPTION OF PREFERRED EMBODIMENTS

The present invention relates to immunomodulatory compositions comprising extracts from medicinal fungi, particularly medicinal mushrooms, and the use of such compositions to provide one or more health benefits and/or one or more immunostimulatory responses in an animal subject.

Medicinal mushrooms comprise a variety of bioactive compounds, including polysaccharides, polysaccharopeptides, phenolic compounds, proteins, lipid components and terpenoids, including triterpenoids such as ganoderic acids. As described herein, mushroom polysaccharides, particularly β-D-glucans, are considered by the applicants, without wishing to be bound by any theory, to be at least in part responsible for some of the immunomodulatory effects associated with certain medicinal mushrooms.

Approximately 80% of the mushroom cell wall consists of polysaccharides, a large proportion of which are glucans. Glucans are the common name given to a group of chemically heterogeneous glucose (Glc) based polysaccharides that are classified based on the nature of the glycosidic linkage as either α- or β-glucans. β-Glucans have a common structure comprising a main chain of β-(1,3)- and/or β-(1,4)-D-Glc units, along with side chain Glc of varying lengths and linkage. Fungal β-glucans comprise β-(1,6)-glucopyranosidic side chains, and particularly branched (1,3:1,6) β-glucans.

Mushroom extracts comprising β-glucans, including for example extracts comprising more than about 10% w/w β-glucans, are particularly contemplated.

In certain embodiments, mushroom extracts comprising triterpenoids, such as for example one or more ganoderic acids, are used. For example, in one embodiment mushroom extracts comprising more than about 1% w/w ganoderic acid, more than about 1.5% w/w ganoderic acid, or more than about 2% w/w ganoderic acid, are used.

Mushroom extracts comprising polysaccharides such as β-glucans can be prepared using techniques well known in the art, and can include or be derived from the whole or any part of the fungal mass, including fruiting bodies, mycelium, spores, or sclerotia.

Mushrooms for use in the preparation of extracts and compositions as described herein may be gathered from the wild and/or cultivated. For example, cultivated mushrooms are grown on certified organic and biodynamic brown rice. In general, the compositions described herein comprise extracts from two or more species of mushroom-derived from one or more parts of the mushroom, such as the mycelia, extracellular components in the mycelium biomass, fruiting bodies, and spores from the fruiting body. In one embodiment, the composition comprises at least one extract derived from the fruiting bodies, spores, and mycelium of one or more mushrooms, and at least one extract derived from the fruiting bodies from one or more mushroom species.

In some embodiments, when mycelium is used to prepare an extract then extracellular components in the mycelium biomass may also be present in the extract or composition, or other components, for example from the growth media, may be present. Extracellular components in the mycelium biomass may arise from sources selected from the group consisting of (a) components produced by the mushroom, (b) components produced by other organisms (non-limiting examples include microbes, plants, animalia, or other fungi) present in or near the mycelium biomass, (c) components naturally present in the mycelium biomass (non-limiting examples include minerals or vitamins naturally present in the soil in which the mycelium grows), and (d) components produced during the growth of the mushroom.

In certain embodiments, one or more of the extracts, such as each of the extracts, is derived from the fruiting bodies of the mushroom species.

In one embodiment, the composition comprising a combination of two or more mushrooms or components or extracts derived from two or more mushrooms selected from the group consisting of Reishi, Maitake, Shiitake, Lion's Mane, Cordyceps, Mesima, Turkey Tail, and Chaga.

Reishi species (for example, Ganoderma lucidum) have been used as a medicine in China and Japan for over 4,000 years. Compositions contemplated herein will in certain embodiments contain extracts derived from reishi species, for example from the mycelium, from the fruiting bodies, or from the mycelium and fruiting bodies. In one embodiment, suitable extracts are prepared from reishi grown under controlled cultivation methods, such as, for example, by growing on rice as well as additional fruiting bodies and spores that have been grown on wood pulp.

Shiitake species (for example, Lentinula edodes) have been cultivated in China and Japan for approximately a thousand years. Compositions contemplated herein will in certain embodiments contain extracts derived from shiitake species, for example from the mycelium, from the fruiting bodies, or from the mycelium and fruiting bodies. In one embodiment, suitable extracts are prepared from shiitake grown under controlled cultivation methods, such as, for example, by growing on rice as well as additional fruiting bodies and spores that have been grown on solid media.

Lion's Mane species (for example, Hericium erinaceus) has been used in traditional herbal systems to promote good digestion, general vigor, strength and nutrition. Compositions contemplated herein will in certain embodiments contain extracts derived from Lion's mane species, for example from the mycelium, from the fruiting bodies, or from the mycelium and fruiting bodies.

Cordyceps species (for example, Cordyceps sinensis) is highly regarded in China as a recovery tonic. Compositions contemplated herein will in certain embodiments contain extracts derived from Cordyceps species, for example from the mycelium, from the fruiting bodies, or from the mycelium and fruiting bodies.

Maitake species (for example, Grifola frondosa) is native to the northeastern part of Japan and to North America, and is used in traditional Chinese and Japanese herbology to support the immune system. Compositions contemplated herein will in certain embodiments contain extracts derived from maitake species, for example from the mycelium, from the fruiting bodies, or from the mycelium and fruiting bodies.

Poria species (for example, Poria cocos) is widely used in traditional Chinese herbalism, often for treating insomnia, restlessness, fatigue, sleep disorder, tension, and nervousness. Compositions contemplated herein will in certain embodiments contain extracts derived from Poria species, for example from the mycelium, from the fruiting bodies, or from the mycelium and fruiting bodies.

Mesima species (for example, Phellinus linteus) has been used in herbal systems in Korea for immune support and as an anti-cancer agent. Compositions contemplated herein will in certain embodiments contain extracts derived from Meisma species, for example from the mycelium, from the fruiting bodies, or from the mycelium and fruiting bodies.

Turkey tail (Coriolus) species (for example, Trametes versicolor) has been a component of traditional Asian medicine for centuries. Compositions contemplated herein will in certain embodiments contain extracts derived from Coriolus species, for example from the mycelium, from the fruiting bodies, or from the mycelium and fruiting bodies.

Chaga species (for example, Inonotus obliquus) has been used in Eastern European botanical medicine since about the 16th century. Compositions contemplated herein will in certain embodiments contain extracts derived from Chaga species, for example from the sclerotia, mycelium, from the fruiting bodies, or from the sclerotia, mycelium and fruiting bodies.

In various embodiments, the compositions herein comprise extracts derived from the fruiting bodies of Reishi, Shiitake, and Maitake mushrooms, or are enriched in extracts from the fruiting bodies of Reishi, Shiitake, and Maitake mushrooms.

Suitable extracts or raw materials suitable for making such extracts by methods well known in the art, including aqueous or alcohol/aqueous extraction methods, are commercially available, for example from Nammex (Box 1780, Gibsons, BC, Canada VON 1V0), Garuda International (PO Box 159, Exeter, CA 93221-0159, USA), Aloha Medicinals (2300 Arrowhead Dr, Carson City, Nev. 89706, United States), Huisong Pharmaceuticals (236 N Jianguo Rd 15F, Hangzhou, Zhejiang 310003, China), Zhejiang Fangge Pharmaceutical Co., Ltd. (Qingyuan, Lishui, Zhejiang, China), or Fungi Health (32568 Williams Ave, Mission, BC V2V 2H1, Canada). Those skilled in the art will appreciate that other suppliers of suitable raw materials or extracts can be selected using the disclosure and examples presented herein.

Various methods to prepare extracts from fungi, including from fungi fruiting bodies, are well known in the art, including for example solvent extraction, ultrasonic extraction, high pressure extraction, and supercritical extraction, such as those described in US patent application U.S. Ser. No. 14/387,392, published as US20150099871A1, incorporated herein by reference in its entirety.

In one embodiment, the extract is an aqueous extract. In one embodiment the extract is an hydroethanolic extract.

In one embodiment, the Maitake extract is Maitake (Grifola frondosa) Mushroom Extract 4:1 Certified Organic (Nammex Product Code MAIEXT4-O).

In one embodiment, the Reishi extract is Red Reishi (Ganoderma lucidum) Mushroom Extract Standardized Certified Organic (Nammex Product Code REIEXT16-0).

In one embodiment, the Shiitake extract is Shiitake (Lentinula edodes) Mushroom Extract Certified Organic (Nammex Product Code SHIEXT4-O).

In certain embodiments, compositions are administered to deliver from about 0.1 g dry equivalent to about 10 g dry equivalent of each fungi per day to an adult human subject. For example, solid or liquid compositions are administered to deliver from about 0.1 g to about 10 g dry equivalent of fungal fruiting bodies from each fungi per day.

In certain embodiments, compositions are administered to deliver from about 0.2 g dry equivalent to about 10 g dry equivalent of each fungi per day to an adult human subject, for example from about 0.3 g to about 10 g, from about 0.4 g to about 10 g, from about 0.5 g to about 10 g, from about 0.6 g to about 10 g, from about 0.7 g to about 10 g, from about 0.8 g to about 10 g, from about 0.9 g to about 10 g, from about 1 g to about 10 g, from about 1 g to about 9 g, from about 1 g to about 8 g, from about 1 g to about 7 g, or from about 1 g to about 6 g dry equivalent of each fungi per day to an adult human subject. For example, solid or liquid compositions are administered to deliver from about 1.5 g to about 10 g dry equivalent of fungal fruiting bodies from each fungi per day.

For example, compositions are administered to deliver from about 2 g dry equivalent to about 10 g dry equivalent of each fungi per day to an adult human subject. For example, solid or liquid compositions are administered to deliver from about 2 g to about 10 g dry equivalent of fungal fruiting bodies from each fungi per day.

In one exemplary embodiment, the composition is formulated so that the daily dose provides from about 4 g to about 6 g dry equivalent Maitake fruiting body, from about 5 g to about 7 g dry equivalent Reishi fruiting body, and from about 5 g to about 7 g dry equivalent Shiitake fruiting body.

In one embodiment, the composition is a liquid composition. Liquid compositions include solutions, suspensions, and emulsions. In one example, the liquid composition comprises water, for example for oral administration, or water or water/propylene glycol solutions for parenteral injection. Liquid preparations can also be formulated in solution in aqueous polyethylene glycol solution. Aqueous solutions suitable for oral use can be prepared by dissolving the active ingredient(s) in water and optionally adding suitable colorants, flavors, stabilizers and thickening agents as desired. Aqueous suspensions suitable for oral use can be made by, for example, dispersing a finely ground mushroom extract or component in water with a viscous material, such as natural or synthetic gums, resins, methylcellulose, sodium carboxymethylcellulose, and other well-known suspending agents.

In certain embodiments, liquid compositions are administered to deliver from about 1 g dry equivalent to about 5 g dry equivalent of each fungi per dose. For example, liquid compositions are administered to deliver from about 1 g to about 5 g dry equivalent of fungal fruiting bodies from each fungi per dose.

In a specifically contemplated embodiment, a liquid composition is formulated so that a single dose of the liquid composition comprises from about 2 g to about 3 g dry equivalent Maitake fruiting body, from about 2.5 g to about 3.5 g dry equivalent Reishi fruiting body, and from about 2.5 g to about 3.5 g dry equivalent Shiitake fruiting body.

In certain embodiments, liquid compositions are administered to deliver from about 2 g dry equivalent to about 10 g dry equivalent of each fungi per day. For example, liquid compositions are administered to deliver from about 2 g to about 10 g dry equivalent of fungal fruiting bodies from each fungi per day.

In another specifically contemplated embodiment, a liquid composition is formulated so that the daily dose of the liquid composition provides from about 4 g to about 6 g dry equivalent Maitake fruiting body, from about 5 g to about 7 g dry equivalent Reishi fruiting body, and from about 5 g to about 7 g dry equivalent Shiitake fruiting body.

In another embodiment, the composition is a solid preparation or dosage form, such as a powder, tablet, dispersible granule, capsule, cachet, or suppository.

In various embodiments, the compositions described herein comprise one or more pharmaceutically acceptable carriers, excipients, or diluents, as required.

A carrier can be one or more substances which may also act as diluents, flavoring agents, solubilizers, lubricants, suspending agents, binders or tablet disintegrating agents. It can also be an encapsulating material. In powders, the carrier will typically be a finely divided solid which is in admixture with the active ingredients. In tablets, the actives are mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, microcrystalline cellulose, silicas including colloidal silicas, glycollates, low melting point wax, cocoa butter, and the like.

Solid dosage forms are in certain embodiments formulated to provide equivalent daily doses to those described above, whether in single or divided dosage forms (such as multiple tablets). Those skilled in the art will appreciate that multiple tablets may be required to deliver certain doses and achieve the required patient compliance.

The term “preparation” is intended to include the formulation of the one or more active ingredients with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets may be provided in chewable form, using techniques well known in the art.

In various embodiments, the composition is a liquid product as set out below:

| | | Equivalent | |
|---|---|---|---|
| | Extract | dry mushroom | |
| Active ingredient | mg/mL | mg/mL | Ratio |
| Grifola frondosa (Maitake) mushroom | 83 | 332 | 1.0 |
| fruiting body; aqueous, dry, | | | |
| concentrated extract, DER* 4:1 | | | |
| Ganoderma lucidum (Reishi) mushroom | 25 | 400 | 1.2 |
| fruiting body; hydroethanolic, dry, | | | |
| concentrated extract, DER* 16:1 | | | |
| Lentinula edodes (Shiitake) mushroom | 100 | 400 | 1.2 |
| fruiting body; aqueous, dry, | | | |
| concentrated extract, DER* 4:1 | | | |

*DER: drug-extract ratio (w/w).

In another embodiment, the composition is a tablet as set out below:

| | | Equivalent | |
|---|---|---|---|
| | Extract | dry mushroom | |
| Active ingredient | mg/tab | mg/tab | Ratio |
| Grifola frondosa (Maitake) mushroom | 208.34 | 833.36 | 1.0 |
| fruiting body; aqueous, dry, | | | |
| concentrated extract, DER* 4:1 | | | |
| Ganoderma lucidum (Reishi) mushroom | 62.50 | 1000.00 | 1.2 |
| fruiting body; hydroethanolic, dry, | | | |
| concentrated extract, DER* 16:1 | | | |
| Lentinula edodes (Shiitake) mushroom | 250.00 | 1000.00 | 1.2 |
| fruiting body; aqueous, dry, | | | |
| concentrated extract, DER* 4:1 | | | |

As used herein, the terms “administer,” “administering,” and “administration,” refer to any method which, in sound medical practice, delivers the composition to a subject in such a manner as to provide a therapeutic effect, and may include implanting, absorbing, ingesting, injecting, inhaling, or otherwise introducing a composition has herein described in or on a subject.

The term “modulating” as used herein refers to the process of producing an effect on biological activity, function, health, or condition of a cell or an organism in which such biological activity, function, health, or condition is maintained, enhanced, diminished, or treated, for example in a manner which is consistent with the normal function of the cell or the general health and well-being of the organism.

As used herein, the phrases an “effective amount” or a “therapeutically effective amount” of a composition, or of an active agent or ingredient or pharmaceutically active agent or ingredient (which are synonymous herein), refer to an amount of the composition or pharmaceutically active agent sufficient enough to have a therapeutic effect upon administration. A therapeutically effective amount of the composition or pharmaceutically active agent may, will, or is expected to elicit a response, such as an immunomodulatory response, and/or provide a health benefit or therapeutic effect, and/or cause a relief of symptoms, and/or effect a treatment. Effective amounts of the composition or pharmaceutically active agent will vary with the particular condition or conditions being treated or the health benefit to be achieved, the severity of the condition, the duration of the treatment, the specific components of the composition being used, and like factors.

The term “enhancing” the biological activity, function, health, or condition of a cell or an organism refers to the process of augmenting, fortifying, strengthening, or improving.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals, including primates, commercially relevant mammals (such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is non-human animal. In certain embodiments, the animal is fish.

A “patient” as used herein will typically refer to a human subject in need of treatment of a disease.

As used herein, the terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

As used herein, the terms “condition,” “disease,” and “disorder” are used interchangeably. As used herein, a “treatment” or “treating” of a disease, disorder, or condition encompasses alleviation of at least one symptom thereof, a reduction in the severity thereof, or the delay, prevention, or inhibition of the progression thereof. Treatment need not mean that the disease, disorder, or condition is totally cured. A composition useful in treatment as contemplated herein needs only to reduce the severity of a disease, disorder, or condition, reduce the severity of symptoms associated therewith, provide improvement to a patient or subject's quality of life, or delay, prevent, or inhibit the onset of a disease, disorder, or condition.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Wherein the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the scope of the invention.

### EXAMPLES

### Example 1

This example describes the analysis of various medicinal mushroom extracts, the preparation of mushroom extract-containing compositions, and analyses of the immunomodulatory effects of such extracts and compositions.

Methods

Mushroom Preparations

Mushroom preparations, including aqueous extracts of fruiting bodies and preparations containing mycelium as well as fruiting bodies, were obtained from various commercial suppliers and outlets, as shown in Table 1 below. A composition (referred to herein as M18-13, and in various figures herein as “formula”) comprising a combination of fruiting body extracts from Reishi (M18-1, 10 parts), Maitake (M18-2, 33 parts), and Shiitake (M18-3, 40 parts) mushrooms was also prepared.

TABLE 1
Commercially available mushroom extracts used

| Source | Mushroom part | Ref ID | Extract ratio |
|---|---|---|---|
| Reishi | Fruiting body | 1632 | 10:1 |
| Reishi | Fruiting body, mycelium, | 1622 | WM |
| | growth medium | | |
| Reishi | Fruiting body | 1628 | 4:1 |
| Reishi | Fruiting body, mycelium, | 1639 | WM |
| | growth medium | | |
| Reishi | Fruiting body, mycelium | 1631 | 66:1 |
| Shiitake | Fruiting body, mycelium, | 1634 | WM |
| | growth medium | | |
| Shiitake | Fruiting body | 1630 | 4:1 |
| Shiitake | Fruiting body | 1633 | 4:1 |
| Maitake | Fruiting body | 1629 | 4:1 |
| Maitake | Fruiting body, mycelium, | 1635 | WM |
| | growth medium | | |
| Reishi | Fruiting body | M18-1 | 16:1 |
| Maitake | Fruiting body | M18-2 | 4:1 |
| Shiitake | Fruiting body | M18-3 | 4:1 |
| Formula: | Fruiting bodies | M18-13 | * |
| Reishi | | M18-1 | 16:1 (10 parts) |
| Maitake | | M18-2 | 4:1 (33 parts) |
| Shiitake | | M18-3 | 4:1 (40 parts) |

WM: Whole mushroom
* On a dry weight equivalent basis, the Maitake:Reishi:Shiitake ratio is 1.0:1.2:1.2.

Quantification of α- and β-Glucans in Mushroom Extracts

α- and β-Glucan content was measured using the Megazyme kit K-YBGL (Megazyme Inc. IL. USA), according to the manufacturer's instructions.

Measurement of α-glucan (starch/glycogen)—Approximately 100 mg of the sample was added to a 20×125 mm screw capped tube, and the tube was tapped to ensure that the entire sample fell to the bottom of the tube. A magnetic stirrer bar and 2.0 mL of ice-cold 2 M KOH was added to each tube, and the tube contents were stirred using a magnetic stirrer in an icewater bath for 20 min to dissolve the starch/glycogen. 1.2 M sodium acetate buffer (pH 3.8; 8 mL) was added to each tube with mixing on a vortex stirrer. Amyloglucosidase (1630 U/mL) plus invertase (500 U/mL) (200 μL) (from Megazyme kit) was immediately added, the contents were mixed well, and the tubes were incubated at 40° C. for 30 min. This solution (10.3 mL final volume) was centrifuged at 1500 rpm for 10 min and 0.1 mL of the supernatant solutions was analyzed for glucose with glucose oxidase/peroxidase reagent.

Measurement of total glucan—Approximately 100 mg (weighed accurately) of the sample was added to a 20×125 mm screw capped tube, and the tube was tapped to ensure that the entire sample fell to the bottom of the tube. A total of 2.0 mL of ice-cold 12 M sulfuric acid was added to each tube, and the tubes were capped and stirred on a vortex mixer. Tubes were placed in an ice-water bath and left for 2 h. During this time, the tube's contents were vigorously stirred (for 10-15 s) several times on a vortex mixer to ensure complete dissolution/dispersion of the sample. Water (2×5 mL) was added in two portions to each tube, and the tubes were capped and vigorously stirred on a vortex mixer for 10 s. The caps were loosened and the tubes were placed in a hot-block heater (˜100° C.). After 5 min, the caps were tightened and the incubation was continued at 100° C. for 2 h. The tubes were cooled to room temperature, and the caps were carefully loosened. 10 M KOH (6 mL) was added, and the tube contents were mixed well. The contents of each tube were quantitatively transferred to 100 mL volumetric flasks using 200 mM sodium acetate buffer (pH 5), and the volume was adjusted to 100 mL with 200 mM sodium acetate buffer (pH 5). The contents were mixed thoroughly, and an aliquot (˜10 mL) of the solution was centrifuged 1500 rpm for 10 min in a bench centrifuge. 100 μL of the sample solution was incubated with 100 μL of a mixture of exo-1,3-β-glucanase (20 U/mL) plus β-glucosidase (4 U/mL) at 40° C. for 60 min, and the glucose was determined with GOPOD reagent as previously described (all of the reagents used were in the Megazyme kit). Absorbance was measured at 510 nm. Concurrently, a 0.1 mL aliquot of glucose standard solution (1 mg/mL), was incubated in quadruplicate (standard) with GOPOD reagent; also, 0.1 mL of acetate buffer (200 mM, pH 5) was incubated with 3.0 mL of GOPOD reagent (reagent blank).

Determination of β-glucan—The β-glucan content was determined by subtracting the α-glucan content from the total glucan content.

Macrophage Cell Model

Human peripheral blood from healthy human volunteers was collected in lithium heparin vacuettes (Wishmed, NSW, AUS) and inverted 5 times to prevent coagulation. Initially 15 mL of Histopaque®-1077 (Ficoll solution (1.077 g/mL), Sigma-Aldrich Co. LLC, MO, USA) solution at room temperature was added into two 50 mL centrifuge tubes and 20 mL-25 mL of peripheral blood was layered on top carefully without disturbing the Histopaque layer. Both tubes were centrifuged at 400 g without brake for 30 minutes at room temperature to generate a density gradient. First the layer of plasma was aspirated out and discarded, subsequently the layer of peripheral blood mononuclear cells (PBMC) as far into the Histopaque layer without disturbing the red blood cells or neutrophils was collected and placed into a fresh tube. The PBMCs were then washed with Roswell Park Memorial Institute medium (RPMI) 1640 medium (Thermo Fisher Scientific, MA, USA) in a 1:1 solution and centrifuged for 10 minutes at 300 g, 10° C. with the brake on. The resulting supernatant was discarded and the RPMI wash step repeated with 10 mL of RMPI under the same conditions. Again, supernatant was discarded and the pellet resuspended in culture medium consisting of RPMI with 1% penicillin/streptomycin, 2.5 ng/mL M-CSF (macrophage colony-stimulating factor, Thermo Fisher Scientific, MA, USA) and 10% NBCS (Newborn Calf Serum, Thermo Fisher Scientific, MA, USA). Cells were incubated in Greiner 24 Well TC Plate (Interpath Services, VIC, AUS) for 14 days at 37° C. with 5% CO₂ at a density of 2×10⁵ cells/mL to allow the cells to adhere prior to any experiments being conducted.

Cytokine Expression Analysis

Cells were treated with fresh media containing varying concentration of mushroom extracts or formula (1, 10, 100, 1,000 μg/mL) with or without the addition of 1 μg/mL of LPS and incubated for an additional 72 hours (24 hours for determination of TNF-α). Subsequently cell culture supernatant was collected and stored at −80° C. The cytokine level present in this supernatant was determined using ELISA-based assays supplied by elisakit.com (Melbourne, Australia,). The protocol (Protocol A) used for the ELISA assays was provided in the kit and can also be found at www.elisakit.com/wp-content/themes/elisa/datasheets/0012%20121010%20H%20IL-6.pdf. Briefly the concentration of cytokine present in the cell culture supernatant (samples and controls) was determined using a standard curve produced from the ELISA kit, as described by the manufacturer.

Macrophages express cytokines in response to stimulation, with the size of the response being related to the exposure time and concentration. Half maximal effective concentration (EC50) is a measurement of the concentration of a drug that gives half-maximal response over an exposure time. In the context of this study EC50 represents the half-maximal cytokine stimulatory response induced by the mushroom extracts or compositions over an exposure time. Conversely, half-maximal inhibitory concentration (IC50) is the concentration of an inhibitor that reduces by half the response over an exposure time. In the context of this study IC50 represents the half-maximal cytokine inhibition induced by the mushroom extracts and formula over an exposure time. EC50 and IC50 are key concepts to pharmacology with both being commonly used as a measure of a drug's potency; the lower the EC50 or IC50 the more potent the drug. The typical biological response of an EC50 is a sigmoidal function, with the inflection point at which the increase in response with increasing ligand concentration begins to slow being the EC50, also seen as the halfway point between the baseline and maximal response of the curve. This investigation was undertaken to determine the EC50 or IC50 values of four mushroom extracts and one mushroom formula on the expression of cytokines IL-1α, IL-6, IL-10 and TNF-α in human macrophages with and without LPS stimulation.

Statistical Analysis

Data generated from the ELISA assays were used to produce a Nonlinear Regression Curve performed using GraphPad Prism version 7.03 (Windows, GraphPad Software, La Jolla California USA) to determine the EC₅₀/IC₅₀ values of the mushroom extracts and mushroom compositions. The parameters of the slope utilised was a variable slope with least squares (ordinary) fit and interpolate unknowns from a standard curve, generated with the following equation:

Y=Bottom+(Top−Bottom)/(1+10\hat{ }((Log EC₅₀−X)*HillSlope)).

A response additivity plot was generated to determine the additive effect of the mushroom formula. The additive line was obtained by summing the cytokine expression levels that were proportional to the percentage of extracts used in the mushroom formula. A response above this additive line indicates a synergistic effect between the extracts present in the composition, and beneath the line an antagonistic effect. Another dose-effect curve approach, curve-shift analysis, was also generated from the additive effect of the extracts on the individual dose-effect curves, an example of two extracts can be expressed using the equation, Effect(x+y)=Ex(x+x_{y})=E_{Y}(yₓ+y)=E_{XY}, where Ex is the sum of the dose-effect from extract x plus the effect of extract y at the same dose as x. This curve allows for identifying synergy in two ways: increase in potency, and/or an increase in efficacy relative to the additive extract response.

### Results and Discussion

The determination of α- and β-glucan content in commercially available mushroom extracts using the Megazyme method found that six of the mushroom powders contain 10-20% α-glucan (see Table 2 below). This high level of α-glucans is not typically naturally found in mushrooms and likely indicates the presence of non-mushroom glucans, such as those found in growth substrate (e.g. brown rice, potato starch) and/or excipients (e.g. maltodextrin). The presence of glucans from extraneous sources changed the overall glucan profile as revealed by the changes in β-glucan:total glucan ratio (see Table 2). The compositions comprising Reishi fruiting body extract (M18-1, 10 parts), Shiitake fruiting body extract (M18-3, 40 parts) and Maitake fruiting body extract (M18-2, 33 parts) was found to have a β-glucan:α-glucan ratio of 6.9 and a β-glucan:total glucan ratio of 0.87.

TABLE 2
β-and α-Glucan composition of commercially
available mushroom extracts used

| | | β- | α- | Total | Ratio of | Ratio of |
|---|---|---|---|---|---|---|
| | | glucan | glucan | glucan | β-:α- | β-:total |
| Source | Ref ID | (%) | (%) | (%) | glucan | glucan |
| Reishi | 1632 | 55.4 | 15.1 | 70.5 | 3.7 | 0.79 |
| Reishi | 1622 | 38.6 | 12.6 | 51.2 | 3.1 | 0.75 |
| Reishi | 1628 | 38.0 | 1.1 | 39.1 | 34.5 | 0.97 |
| Reishi | 1639 | 31.0 | 17.8 | 48.8 | 1.7 | 0.64 |
| Reishi | 1631 | 21.4 | 13.9 | 35.3 | 1.5 | 0.61 |
| Shiitake | 1634 | 36.7 | 15.3 | 52.0 | 2.4 | 0.71 |
| Shiitake | 1630 | 12.3 | 1.2 | 13.5 | 10.0 | 0.91 |
| Shiitake | 1633 | 10.2 | 0.8 | 10.9 | 13.6 | 0.93 |
| Maitake | 1629 | 18.6 | 1.3 | 19.9 | 14.9 | 0.94 |
| Maitake | 1635 | 40.6 | 13.3 | 53.9 | 3.1 | 0.75 |
| Reishi | M18-1 | 17.7 | 3.2 | 20.9 | 5.5 | 0.85 |
| Maitake | M18-2 | 32.0 | 3.8 | 35.8 | 8.4 | 0.89 |
| Shiitake | M18-3 | 20.1 | 0.8 | 20.9 | 25.1 | 0.96 |
| Formula | M18-13 | 22.1 | 3.2 | 25.3 | 6.9 | 0.87 |

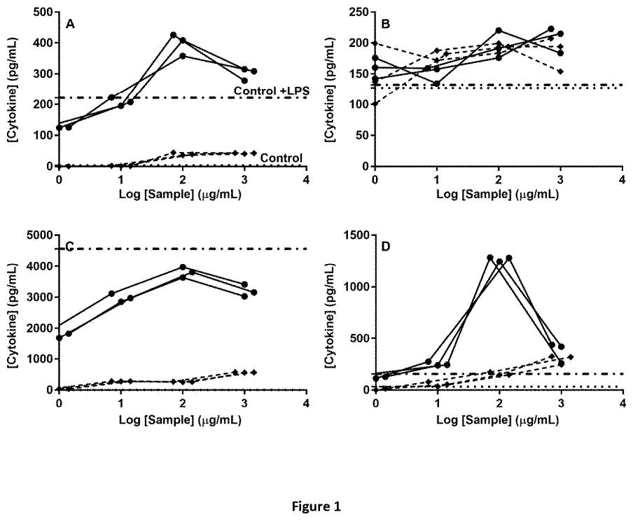

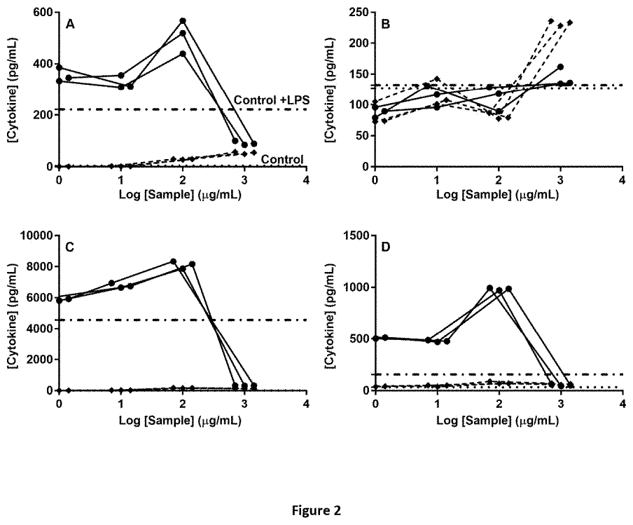

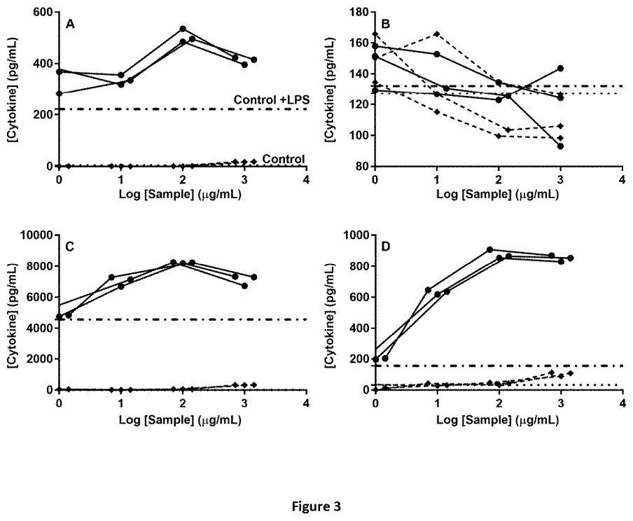

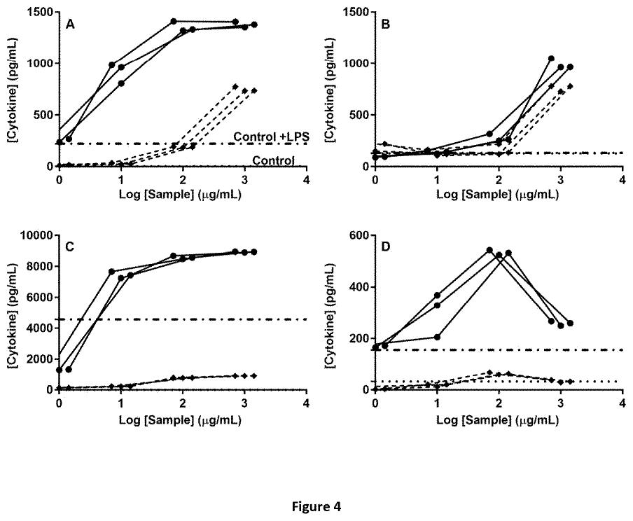

FIGS. 1 to 4 present the results of triplicate repeat experiments conducted on the mushroom extracts and the multi-extract composition to determine the cytokine expression of ±LPS stimulated human macrophages at different concentrations. The majority of mushroom extracts induced dose-dependent increases in cytokine expression (see FIGS. 1-3), except for Maitake (M18-2), which produced a dose-dependent decrease in IL-1α response in both non-LPS and LPS-stimulated macrophages which was decreased with increasing extract concentration (FIG. 3B), and Shiitake (M18-3) which inhibited IL-6 expression in LPS stimulated macrophages at all concentrations (FIG. 1C). Conversely, Shiitake stimulated IL-6 expression in non-LPS treated macrophages (FIG. 1C). The multi-extract composition (M18-13) induced dose-dependent increases in cytokine expression, with a stimulation of cytokine expression induced by the composition observed in both non-LPS and LPS treated macrophages (FIG. 4).

The data presented in FIGS. 1-4 show that, in general, an increase in the concentration of mushroom extract or composition administered corresponded with an increase in cytokine expression up to a peak of 100 or 1,000 μg/mL, depending on the cytokine under investigation.

Comparisons within the data presented in FIG. 4 show that the multi-extract composition induces a greater response of cytokine expression compared to the LPS stimulated control, indicating that the multi-extract composition is immunostimulatory.

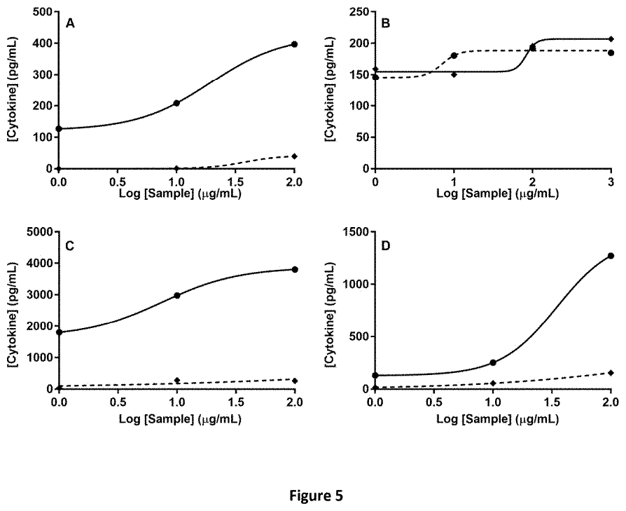

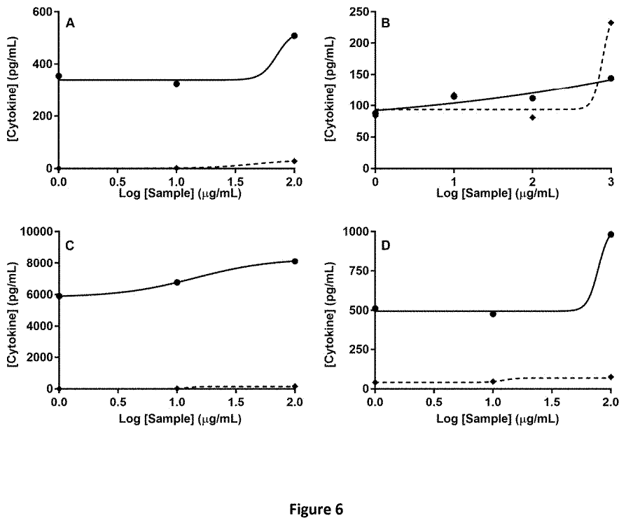

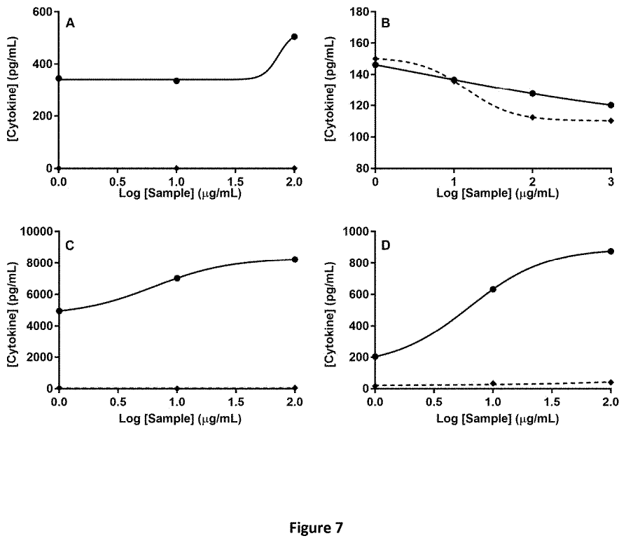

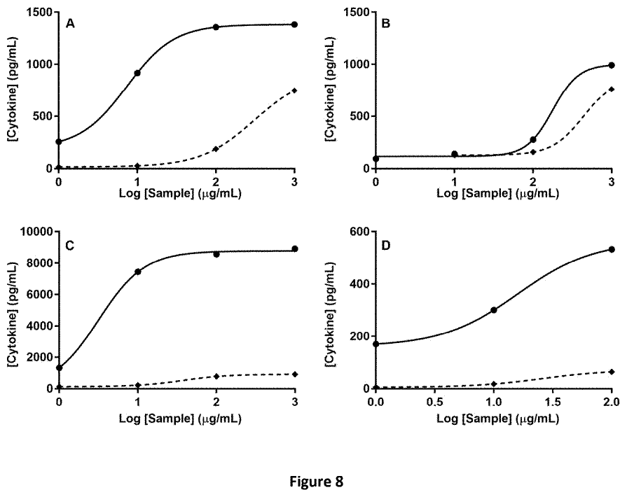

To further explore the immunostimulatory effects of the multi-extract composition and the various individual extracts, EC50 and IC50 values were determined as appropriate. Table 3 provides the EC50 and IC50 values for the individual mushroom extracts, as well as the multi-extract composition (“formula”, in bold) as determined by a nonlinear regression variable slope (see FIGS. 5-8). The majority of the EC50 values were in the low μg/mL range (<100 μg/mL), with the multi-extract composition EC₅₀ values lower in the LPS stimulated macrophages compared to non-stimulated counterparts (see Table 3 and FIG. 8). The exposure time of extracts and multi-extract composition on the macrophages varied, with the TNF-α experiment having the shortest exposure of 24 hours compared to 72 hours exposure for IL-1α, IL-6 and IL10. The shorter exposure time of macrophages to mushroom extracts for TNF-α was due to the relatively quick degradation of TNF-α in cell culture serum as opposed to the other cytokines.

TABLE 3
EC₅₀ and IC₅₀ values associated with the effect of mushroom
extracts and multi-extract composition on cytokine expression
in non-LPS (−) and LPS (+) stimulated human macrophages

| | EC₅₀ or IC₅₀* (μg/mL) | | | | | | | |
|---|---|---|---|---|---|---|---|---|
| | Shiitake | | Reishi | | Maitake | | Formula\hat{ } | |
| | (M18-3) | | (M18-1) | | (M18-2) | | (M18-13) | |
| | −LPS | +LPS | −LPS | + LPS | −LPS | +LPS | −LPS | +LPS |
| TNF-α | 39.8 | 10.8 | 98.4 | 899.7 | 196.4 | 34.2 | 308.9 | 7.4 |
| IL-1α | 7.0 | 82.8 | 778.4 | N.D. | 14.4* | 5.4* | 737.8 | 177.7 |
| IL-6 | N.D. | 8.6 | 12.4 | 735 | 142.8 | 7.9 | 36.9 | 3.2 |
| IL-10 | 20.1 | 34.9 | 31.0 | 78.0 | 5.1 | 6.3 | 25.6 | 16.0 |

Data derived from a nonlinear regression variable slope with interpolated unknowns from standard curve (see FIGS. 5-8), EC₅₀ determined from sample concentrations.
N.D. Not Determined as slope was unable to converge.
*Indicates IC₅₀ values.

Within LPS stimulated macrophages, the EC₅₀ value observed with the multi-extract composition was lower compared to the individual mushroom extracts that comprise the formula for the cytokines TNF-α, IL-6, and second lowest for IL-10 (Table 3). A decrease in EC₅₀ values corresponds to an increase in drug potency. These data support a synergistic effect between the individual components that make up the multi-extract composition when present in the composition. Accordingly, this phenomenon was further investigated as described below.

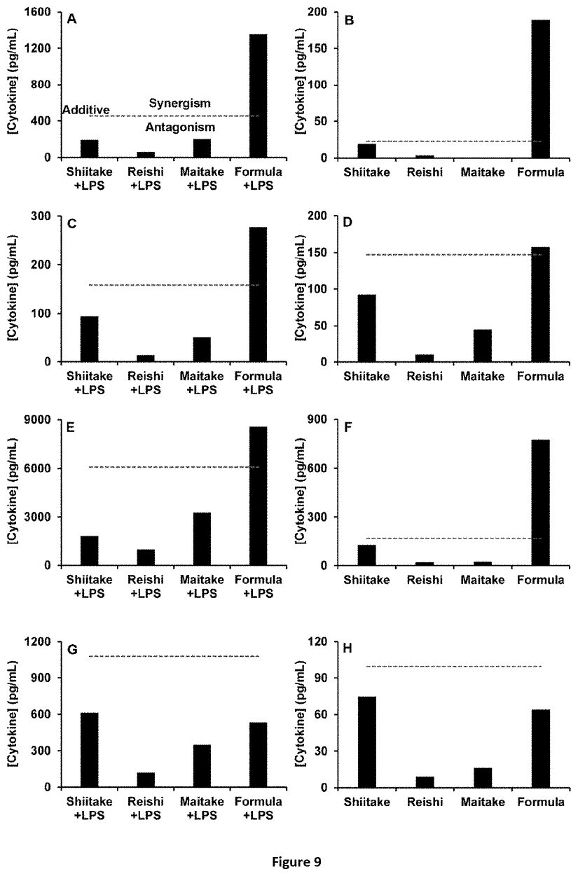

To determine the effect-based responses derived within the mushroom formula, a response additivity graph was constructed (FIG. 9). This illustrates the proportional effect on cytokine expression of the individual extracts comprising the multi-extract composition, in addition to the combination effect of the multi-extract composition itself. Summing the effects of the individual extracts generates an additive line. This line represents the level of expression expected if the response derived from the multi-extract composition was simply the sum of the individual responses of its components. However, the response additivity graph reflects only a drug's efficacy, not its potency. As seen in FIG. 9A, the TNF-α expression for the multi-extract composition in LPS-stimulated macrophages is greater than that of the additive line, indicating a synergistic effect. This synergistic effect was also observed for the remaining cytokines in both ±LPS stimulated human macrophages, in both LPS stimulated and non-LPS human macrophages, except for II-1α in non-LPS stimulated macrophages (FIG. 9D) and IL-10 (FIGS. 9G and 9H).

IL-10 showed a synergistic antagonistic affect where the response from the multi-extract composition was less than that of the summed individual extract responses (FIGS. 9G and 9H). Overall, in combination with the previous observations, these data support the conclusion that not only are the individual mushroom extracts and in particular the multi-extract composition inducing an immunostimulatory response by inducing pro-inflammatory cytokines (IL-1α, IL-6 and TNF-α) and reducing the expression of anti-inflammatory cytokine IL-10, the multi-extract composition is able to do so synergistically.

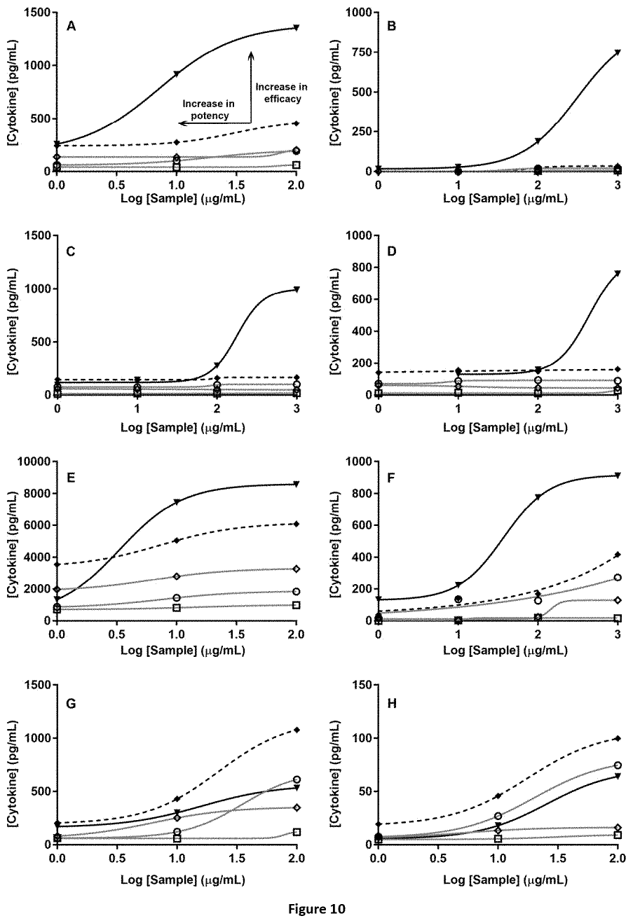

A known limitation inherent to the response additivity design is the assumption that the dose-response curves are linear with a zero intercept. To address these potential limitations with the response additivity graph, a curve-shift analysis was performed (see FIG. 10) which allow the synergistic effect to be measured in two separate ways—an increase in potency (for example, if there is a faster response indicated by inducing a response at lower concentration), and/or an increase in efficacy (for example if there is an overall larger response in the curve relative to the combination effect of the extract responses).

As seen in FIG. 10A for TNF-α, the multi-extract composition (solid black line) demonstrated an increase in both potency and efficacy over the combined effects of the extracts (dashed black line). This indicates that the multi-extract composition induces a faster and a larger cytokine response than that of the individual extracts. This is consistent with the EC50 values, as multi-extract composition-induced TNF-α expression in stimulated macrophages had the lowest EC50 value compared to the individual extracts and therefore highest potency (Table 3) as well as the highest cytokine expression.

The remaining curve-shift analyses (FIGS. 10B-10F) illustrated the synergistic effect observed with the multi-extract composition, with an increase in efficacy as well as an increase in some potency, except for IL-10. The multi-extract composition had an antagonistic effect on IL-10 cytokine expression in both macrophages with LPS stimulation and macrophages without LPS stimulation, with both a lower potency and efficacy (FIG. 10G and FIG. 10H).

As well as being more potent at all concentrations for TNF-α and IL-6 for non LPS treated macrophages (FIGS. 10A, 10B and F), increased potency was also observed with the multi-extract composition at higher concentrations (>100 μg/mL) for IL-1α and IL-6 for LPS treated macrophages (FIGS. 10C, D and E).

### CONCLUSION

The effect on the expression of cytokines IL1-α, IL-6, IL-10 and TNF-α was determined for each mushroom extract and a multi-extract composition. The data presented herein indicates that an immunostimulatory effect is elicited by the majority of mushroom extracts, and by the multi-extract composition, with a dose-dependent increase in pro-inflammatory cytokine expression, and a dose-dependent decrease in anti-inflammatory cytokine expression. The data presented herein further establishes that a synergistic effect on the expression of three proinflammatory cytokines (IL1-α, IL-6 and TNF-α) and a synergistic antagonistic effect on the expression of the anti-inflammatory cytokine IL-10 is observed on administration of the multi-extract composition. These results suggest that the multi-extract composition elicits a strong synergistic immunostimulatory response.

1. An immunomodulatory composition comprising at least one extract from each of two or more medicinal fungi, wherein at least two of the medicinal fungi are Reishi, Shiitake, and/or Maitake and wherein the composition elicits a synergistic immunomodulatory effect.

2. The immunomodulatory composition of claim 1, wherein each of the fungal extracts comprises β-glucan and has a β-glucan:total glucan ratio of at least about 0.6.

3. (canceled)

4. The immunomodulatory composition of claim 1, wherein one or more of the extracts is an aqueous extract or a hydroethanolic extract.

5. The immunomodulatory composition of claim 1, wherein at least one of the extracts is an extract of fungal fruiting bodies or wherein each of the extracts present in the composition is an extract of fungal fruiting bodies.

6. (canceled)

7. The immunomodulatory composition of claim 5, wherein each of the fungal fruiting body extracts is substantially free of mycelia or mycelial extracts.

8. The immunomodulatory composition of claim 4, wherein the composition comprises at least one extract from the fruiting body of Reishi fungus, at least one extract from the fruiting body of Shiitake fungus, and at least one extract from the fruiting body of Maitake fungus.

9. The immunomodulatory composition of claim 4, wherein on a dry weight equivalent basis the Maitake:Reishi ratio is from about 1:4 to about 4:1, from about 1:3 to about 3:1, from about 1:2 to about 2:1, from about 1:1.5 to about 1.5:1, or about 1:1.

10. The immunomodulatory composition of claim 9, wherein on dry weight basis the Maitake:Reishi ratio is about 1:1.2.

11. The immunomodulatory composition of claim 4, wherein on a dry weight equivalent basis the Maitake:Shiitake ratio is from about 1:4 to about 4:1, from about 1:3 to about 3:1, from about 1:2 to about 2:1, from about 1:1.5 to about 1.5:1, or about 1:1.

12. The immunomodulatory composition of claim 11, wherein on dry weight basis the Maitake:Shiitake ratio is about 1:1.2.

13. The immunomodulatory composition of claim 4, wherein on a dry weight equivalent basis the Reishi:Shiitake ratio is from about 1:4 to about 4:1, from about 1:3 to about 3:1, from about 1:2 to about 2:1, from about 1:1.5 to about 1.5:1, or about 1:1.

14. The immunomodulatory composition of claim 13, wherein on dry weight basis the Reishi:Shiitake ratio is about 1:1.

15. The immunomodulatory composition of claim 8, wherein on a dry weight equivalent basis the Maitake:Reishi:Shiitake ratio is 1:1.2:1.2.

16. The immunomodulatory composition of claim 8, wherein the fungal extracts comprise at least 50% w/w of the composition.

17. The immunomodulatory composition of claim 1, wherein the synergistic immunomodulatory effect is a synergistic immunostimulatory effect or an increase in the expression of one or more pro-inflammatory cytokines.

18. (canceled)

19. The immunomodulatory composition of claim 17, wherein the one or more pro-inflammatory cytokines is comprising IL-1, IL-1α, IL-6, and/or TNF-α.

20-21. (canceled)

22. A method of:
modulating the immune system in a subject in need thereof,
eliciting an immune response in a subject;
treating a disease or condition associated with the immune system or of treating a disease or condition associated with immune dysfunction in a subject in need thereof;
conferring a health benefit to a subject in need thereof, wherein the health benefit is selected from the group comprising: supporting healthy immune function, improving convalescence, reducing fatigue, improving respiratory function, reducing coughing, improving immune responses to infectious disease, or decreasing susceptibility to infectious disease; or
reducing susceptibility to one or more infectious diseases in a subject in need thereof;
the method comprising administering to the subject an effective amount of an the immunomodulatory composition of claim 1.

23-30. (canceled)
